(19) United States
(12) Patent Application Publication
(10) Pub. No.: US 2025/0128218 A1
(43) Pub. Date: Apr. 24, 2025

(54) MODULAR SYSTEM FOR AUTOMATED COMPOUNDING APPLICATIONS

(71) Applicant: MEDISCA PHARMACEUTIQUE INC., Saint-Laurent (CA)

(72) Inventor: Panagiota DANOPOULOS, Saint-Laurent (CA)

(73) Assignee: MEDISCA PHARMACEUTIQUE INC., Saint-Laurenet, QC (CA)

(21) Appl. No.: 18/722,076

(22) PCT Filed: Dec. 19, 2022
(86) PCT No.: PCT/CA2022/051859

§ 371 Date: Jun. 20, 2024

Related U.S. Application Data
(60) Provisional application No. 63/291,742, filed on Dec. 20, 2021.

(51) Int. Cl. B01F 33/84; A61J 3/00; B01F 27/232; B01F 35/21; B01F 101/22; G06Q 30/0601

(52) U.S. Cl. CPC B01F 33/8442; A61J 3/00; B01F 27/2324; B01F 33/846; B01F 35/2117; G06Q 30/0635; B01F 2101/22

(57) ABSTRACT

The present disclosure relates to automated compounding system, methods, and devices for manufacturing compounded compositions. The system comprises a controller including a data processor for receiving via data communication over a data network a customer order, the customer order identifying a Prescription ID associated with a compounded composition, a plurality of discrete stations, including a processing station comprising a processing apparatus configured, in response to instructions received from the controller, to perform one or more process steps required for manufacturing the compounded composition, and a plurality of containers, an industrial robot configured, in response to instructions received from the controller, to displace one or more containers from one discrete station to another discrete station.

### CROSS-REFERENCE TO RELATED APPLICATION

The present application claims the benefit of U.S. provisional patent application Ser. No. 63/291,742 filed on Dec. 12, 2021 by Danopoulos, Panagiota. The contents of the above-referenced document are incorporated herein by reference in their entirety.

### TECHNICAL FIELD

This application generally relates to the field of relates to manufacturing and logistics automation equipment system, devices and method. More specifically, the present disclosure relates to an automated system, devices and method for processing customer product orders for manufacturing compounded compositions.

### BACKGROUND

Medical facilities, hospitals, or licensed pharmacists often manufacture compositions that are tailored to the needs of an individual patient or a subgroup of patients, i.e., compounded compositions. Such customized manufacture activities are known as pharmacy compounding, whereby compounding personnel will typically prepare such tailored compositions based on a medical prescription to meet the patient specific needs. Pharmaceutical, cosmetic, or cannabis compositions are non-limiting examples of compounded compositions.

Pharmacy compounding typically involves mixing, melting or grinding ingredients, typically through manual processing using a pestle and mortar, or through magnetic hot plates. However, manual processing can be time-consuming and is often prone to cross-contamination. Along with the contamination risk, there is also the problem that performing manual processing often results in products that face repeatability and/or quality challenges. In other words, it is often difficult to obtain compositions having consistent concentrations of active ingredient from one composition to another and/or consistent homogeneous active ingredient concentration within one preparation per se. This may result in substantial qualitative differences during manufacture of the same recipe, which at minimum can influence the effectiveness of the recipe or in some cases, even render the recipe dangerous to the patient. Further, even when suitable protection measures are taken, leakage and/or drips may occur—either because of human errors or equipment malfunction. These leakages put the compounding personnel at risk of inadvertent exposure to medications/drugs.

In this regard, various practical devices have been previously suggested to overcome the above deficiencies of the manual processing approach to pharmacy compounding.

U.S. Patent Publication 2012/0269029 (Konietzko) describes a program-controlled mixer useful for pharmacy compounding, which includes a control unit, a motor-driven mixing unit with a blade mixing tool, which engages into a mixing vessel, and a lift unit. The lift unit produces an axial relative motion between the blade mixing tool and the mixing vessel, to move the blade mixing tool in the mixing vessel between an upper end position and a lower one, preferably at a constant lifting speed. A deficiency associated with such mixing devices is that they involve mixing using blades that contact the mixture causing high shearing forces, which can generate so much heat during mixing to degrade thermally labile API. Further, the use of blades requires implementing rigorous cleaning procedures after use to reduce risk of cross-contamination.

U.S. Pat. No. 10,993,876 (Danopoulos) describes a program-controlled planetary mixer for pharmacy compounding, which includes a control unit, and a motor-driven mixing unit that imparts superimposed revolution and rotation movements on a container containing ingredients. Such planetary mixer can be used for mixing, grinding, melting, de-aerating, or a combination thereof. Due to the relative dearth of experience in the area, novice users can waste a significant amount of time and product in search of operating parameters that yield satisfactory results. Also, problems may arise due to human error being introduced into the mixing process. Current operation of planetary mixers is also largely reliant upon a user, which increases the probability that human error is introduced either during the mixing or record-making processes.

U.S. Pat. No. 11,130,106 (Danopoulos) describes a program-controlled system for pharmacy compounding, which includes a mixer control device, at least one processor, and a non-transitory storage medium operably connected to the at least one processor and storing computer-readable program instructions implementing a mixer control application. The system implements a computerized graphical user interface (GUI) that provides a user of a computing device with an opportunity to identify a compounding formula; consult a database at least partly on a basis of the identified compounding formula in order to determine processing parameters for a mixer apparatus, associated with the identified compounding formula; and to cause the mixer apparatus to implement movements that result in processing the ingredients in accordance with the parameters determined from consulting the database. While such system addresses the problem of wasting time and product in search of operating parameters, problems may still arise due to human error being introduced, which may occur when selecting and/or handling ingredients.

As such, improvements are needed in current processes and systems for pharmacy compounding.

### SUMMARY

This Summary is provided to introduce a selection of concepts in a simplified form that are further described below in the Detailed Description. This Summary is not intended to identify key aspects or essential aspects of the claimed subject matter.

In one broad non-limiting embodiment, the present disclosure relates to an automated compounding system the system comprising: a controller including a data processor for receiving via data communication over a data network a customer order, the customer order identifying a Prescription ID associated with a compounded composition, a plurality of discrete stations, including a processing station comprising a processing apparatus configured, in response to instructions received from the controller, to perform one or more process steps required for manufacturing the compounded composition, and a plurality of containers, an industrial robot configured, in response to instructions received from the controller, to displace one or more containers of the plurality of containers from one discrete station to another discrete station.

In one broad non-limiting embodiment, the present disclosure relates to a computer-implemented method comprising of manufacturing a compounded composition, the method comprising: receiving a customer order over a data network at a controller that includes a data processor, the customer order identifying a Prescription ID associated with a compounded composition, receiving instructions from the controller at a processing station of a plurality of discrete stations, the processing station comprising a processing apparatus configured, in response to instructions received from the controller, to perform one or more process steps required for manufacturing the compounded composition, and the plurality of discrete stations further comprising a plurality of containers, and receiving instructions from the controller at an industrial robot configured, in response to instructions received from the controller, to displace one or more containers of the plurality of containers from one discrete station to another discrete station.

In one broad non-limiting embodiment, the present disclosure relates to a non-transitory computer-readable medium having instructions in code which when executed by a processor of a controller causes the controller to receive a customer order over a data network, the customer order identifying a Prescription ID associated with a compounded composition, communicate instructions to a processing station of a plurality of discrete stations, the processing station comprising a processing apparatus configured, in response to instructions received from the controller, to perform one or more process steps required for manufacturing the compounded composition, and the plurality of discrete stations further comprising a plurality of containers, communicate instructions to an industrial robot configured in response thereto to displace one or more containers of the plurality of containers from one discrete station to another discrete station.

In some embodiments, any one of the system, the method, and the non-transitory computer-readable medium described herein, includes one or more of the following features, in any permissible combination:
the system has a modular architecture.
the controller implements an ordering logic configured to receive and process the customer order.
the data processor communicates with a database to obtain a computer-readable standard operating procedure (SOP) script associated with the Prescription ID.
the controller implements a product preparation logic configured to define a sequence of operations for manufacturing the compounded composition at least based on the SOP script.
the controller implements an inventory management logic to determine inventory status of the containers.
based on the inventory status determination, the controller determines whether the system is operational to fulfill the customer order.
the plurality of stations includes a feeding station equipped with one or more container support devices configured for holding the plurality of containers.
the plurality of stations includes a loading station for adding ingredients into a container from the plurality of containers.
the industrial robot is configured, in response to instructions received from the controller, to sequentially displace a plurality of ingredient containers from the feeding station to the loading station.
the industrial robot is configured, in response to instructions received from the controller, to pick the container containing the added ingredients and bring the container to the apparatus.
the processing apparatus includes one or more planetary mixer(s).
in response to instructions received from the controller, the processing apparatus is configured to subject ingredients in the container to at least one of mixing, grinding, de-aerating, melting, and any combination thereof.
the plurality of stations includes a scale station equipped with one or more scales to determine the weight of containers.
the plurality of stations includes a labeling station for labeling containers.
the controller includes a machine-readable storage encoded with software for execution by the data processor.
a server arrangement implements the controller.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### DRAWINGS

A detailed description of specific exemplary embodiments is provided herein below with reference to the accompanying drawings in which:

FIG. 1A is a block diagram showing an automated compounding system environment, including an automated compounding system and a system for communicating between a device and a server over a data network, in accordance with an embodiment of the present disclosure.

FIG. 1B is a block diagram showing an automated compounding system environment, including an automated compounding system and a system for communicating between a user interface of the automated compounding system and a server over a data network, in accordance with an embodiment of the present disclosure.

FIG. 2 is a block diagram showing components internal to the server of FIG. 1A or FIG. 1B, in accordance with an embodiment of the present disclosure.

FIG. 3A illustrates a credentials table for storing account information, in accordance with an embodiment of the present disclosure.

FIG. 3B illustrates an SOP table for storing operating procedure information, in accordance with an embodiment of the present disclosure.

FIG. 3C illustrates a table for storing operating procedure information in association with account information, in accordance with an embodiment of the present disclosure.

FIG. 3D illustrates a table for storing ingredient and quantity information in association with account information, in accordance with an embodiment of the present disclosure.

FIGS. 3E and 3F are non-limiting examples of operating procedures, in accordance with an embodiment of the present disclosure.

FIG. 4A is a flowchart illustrating steps that may be taken by an interactive program module to operate the automated system based on a prescription ID, in accordance with an embodiment of the present disclosure.

FIG. 4B illustrates a graphical user interface (GUI) on the device that may appear during execution of the flowchart in FIG. 4A, in accordance with an embodiment of the present disclosure.

FIG. 4C is a flowchart illustrating steps that may be taken by the program module assess whether the system is operational for the obtained SOP, in accordance with an embodiment of the present disclosure.

FIG. 5 is a block diagram showing components of the magazine containing ingredients, in accordance with an embodiment of the present disclosure.

FIG. 6 is a block diagram showing components internal to a mixer contained in the automated compounding system, in accordance with an embodiment of the present disclosure.

FIG. 7 is a block diagram showing entities participating in a validation process, in accordance with an embodiment of the present disclosure.

FIG. 8 illustrates table for storing information about a variety of formulas, in accordance with an embodiment of the present disclosure.

FIG. 9 is a block diagram showing a server, a tag reader, and a tag-enabled container, in accordance with an embodiment of the present disclosure.

FIG. 10 is a block diagram showing the tag reader being coupled to a database containing operating parameters, in accordance with an embodiment of the present disclosure.

FIG. 11 is a flowchart showing steps in a validation process, in accordance with an embodiment of the present disclosure.

FIG. 12 illustrates a graphical user interface (GUI) on the device of FIG. 1A or on the user interface of FIG. 1B that may appear during execution of the flowchart in FIG. 4A, in accordance with an embodiment of the present disclosure.

FIG. 13 is a flowchart of a process for one-click refill, in accordance with an embodiment of the present disclosure.

FIG. 14 illustrates a graphical user interface (GUI) on the device of FIG. 1A or on the user interface of FIG. 1B that may appear during execution of the flowchart in FIG. 13, in accordance with an embodiment of the present disclosure.

FIG. 15 is a flowchart of a process for executing the one-click refill, in accordance with an embodiment of the present disclosure.

FIG. 16 is a flowchart of a process for filtering SOPs, in accordance with an embodiment of the present disclosure.

FIG. 17 is a non-limiting functional block diagram showing components of the compounding system interfacing with a controller, in accordance with an embodiment of the present disclosure.

FIG. 18 is a non-limiting functional block diagram of the controller for operating the compounding system, in accordance with an embodiment of the present disclosure.

FIG. 19 is a non-limiting illustration of the compounding system including an industrial robot for manufacturing a compounded composition, in accordance with an embodiment of the present disclosure.

FIG. 20 is a non-limiting flowchart illustrating a process for ingredient validation.

In the drawings, exemplary embodiments are illustrated by way of example. It is to be expressly understood that the description and drawings are only for the purpose of illustrating certain embodiments and are an aid for understanding. They are not intended to be a definition of the limits of the invention.

### DETAILED DESCRIPTION

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art considering the instant disclosure which variations and additions do not depart from the present technology. Hence, the following description is intended to illustrate some embodiments of the technology, and not to exhaustively specify all permutations, combinations, and variations thereof.

The present inventor has developed an automated system, devices, and method for manufacturing compounded compositions, such as pharmaceutical, cannabis, or cosmetic compositions. In particular, the automated system, devices, and method described herein allow processing customer orders relating to compounded compositions. Advantageously, the system, devices, and method described herein can be implemented with minimal, or in some cases substantially without, human involvement.

The system, devices, and method described herein afford one or more technical advantages as will be apparent to a person skilled in the art in view of the present disclosure.

For example, the system, devices, and method described herein afford relative flexibility in that the system may be customized or expanded according to business needs and/or market changes. For example, the system, devices, and method described herein may facilitate headcount reduction in a compounding manufacturing facility. For example, the system, devices, and method described herein may reduce human errors in the preparation of compounded compositions, increase prescription preparation output rate, and/or increase accuracy of composition concentrations. For example, the system, devices, and method described herein may support inventory management. For example, the system, devices, and method described herein may protect compounding personnel in the handling of medication and/or drugs. For example, the system, devices, and method described herein may lower the cost-per-dose of medication and reduce the need for outsourcing. For example, the system, devices, and method described herein may minimize the risk of medication errors due to human error that can result in patient injury, emergency intervention, extended hospitalizations and liability. For example, the system, devices and method described herein may be tailored to various compounding manufacturing facility configurations, or application type required.

In some embodiments, the compounded composition of the present disclosure can be any one of a cream, ointment, lotion, emulsion, gel, suspension, powder, liquid solution, colloidal dispersion, troche or syrup.

In some embodiments, operation of the system described herein involves sufficient laminar air to ensure adequate airflow to clear the system, ensuring particulate is swept away from critical areas, and provide continues particle monitoring that can stop the process if contamination is detected.

In some embodiments, operation of the system described herein involves sufficient sanitization means, for example with proper positioning and exposure duration to ultra-violet (UV) rays.

In some embodiments, the system, devices and method described herein may ensure compliance with USP<797> and/or <800>, Drug Quality and Security Act (DQSA) regulations, cGMP, tracking standards, or a combination thereof.

### Automated System

Various components and devices of an automated system in accordance with the present disclosure will now be described in further details.

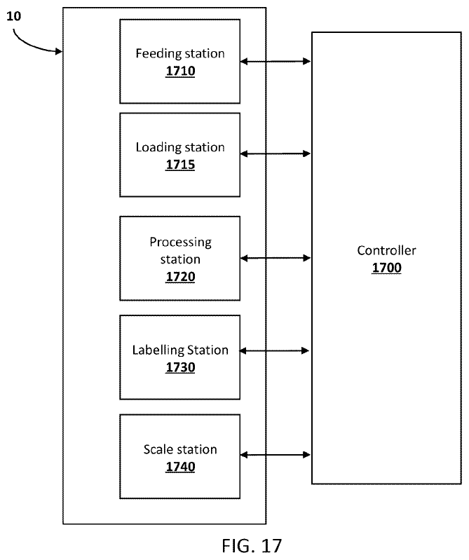

In a non-limiting example of implementation, and with further reference to FIG. 17, the automated compounding system 10 includes a plurality of discrete stations for performing manufacturing steps to obtain a compounded composition.

In some embodiments, the processes performed at respective discrete stations can run simultaneously or in series, depending on specifics of the manufacturing process to obtain the compounded composition being implemented.

In some embodiments, the automated compounding system 10 includes a feeding station 1710 for holding a plurality of containers. The plurality of containers includes empty containers intended for processing the ingredients, or ingredient containers that contain ingredients for manufacturing the compounded compositions, for example. The empty containers can be general mixing purpose containers or can be dispensing containers. For example, dispensing containers include metered-dose containers. Metered-dose containers may include mechanisms for modifying a volume of the container, such as a rod and moving plate (such as the Topi-CLICK™ from DoseLogix, LLC, USA), a piston or pump (such as in a syringe), may be rod-less (such as the UnoDose™ Metered-Dose Topical Applicator, available from Medisca Pharmaceutique, Canada), and the like. Non-limiting examples of dispensing containers are described in for example U.S. Application Publication 2014/0,221,945, US Application Publication 2021/0,299,621, US Application Publication 2018/0,345,305, and US Application Publication 2018/0,125,753, each of which is hereby incorporated by reference into its entirety for all purposes. In use, a metered-dose container allows administrating a dose of compounded composition, the dose being metered by weight or by volume. The container may comprise paper, cardboard, plastic, metal and/or glass materials. The container may be labeled, marked with information, such as a name of a patient, a name of medication, a barcode and/or a moment (i.e. a day, a date and/or a moment of the day) at which the compounded composition is intended for administration. Other examples of containers include jars, bins, vessels, bags, packets, boxes, bottles, and cartridges (for vaporization devices, for example), any of which could be made from wood, paper, cardboard, plastic, glass and/or metal, for example. Some containers, such as jars and bottles, can be sealed with caps or lids.

In some embodiments, the feeding station 1710 includes one or more container support devices configured for holding the plurality of containers. Characteristics of the one or more container support devices will be further explained later in this text.

In some embodiments, the automated compounding system 10 includes a loading station 1715 for adding ingredients into a container. For example, the loading station may include loading means, such as dosing heads for adding controlled volumes of liquids and/or creams, or for adding controlled weights of powder, and the like. Optionally, the loading station 1715 includes one or more code reader for validating the ingredient being added to the container. Characteristics of the one or more code reader will be further explained later in this text.

In some embodiments, the automated compounding system 10 includes a processing station 1720 for processing the ingredients to form the compounded composition. For example, such processing may include mixing, diluting, grinding, melting, de-aerating, heating, cooling, or any combination thereof. For example, the processing station 1720 can be equipped with one or more processing apparatus. For example, for processing ingredients in a container for forming the compounded composition. An example of a suitable apparatus for processing ingredients can be a planetary mixer configured for imparting superimposed rotation and revolution movements on a container. The superimposed rotation and revolution movements eliminate the need for mixing rods, blades and an evacuation device, thus reducing processing times. Examples of suitable planetary mixers are described in U.S. Pat. No. 10,231,903, which is hereby incorporated by reference in its entirety. For example, a suitable non-limiting example of a planetary mixer may be any of the Maz™ mixer models sold by Medisca Pharmaceutique Inc. of St-Laurent, Canada, and/or any of the SpeedMixer™ Smart DAC models sold by Hauschild of Germany, which can interface and can be controlled through a controller (described later in this text). Another example of a mixer can be a stirring tool, as described in U.S. Pat. No. 10,493,413, which is hereby incorporated by reference in its entirety. A suitable non-limiting example of a stirring tool may be any of the Samix stirring tool models sold by Medisca Pharmaceutique Inc. of St-Laurent, Canada, which can interface and can be controlled through a controller. The reader will readily understand that the one or more processing stations 1720 can include other types or models of apparatus for processing ingredients, without departing from the present disclosure.

In some embodiments, the automated compounding system 10 includes a scale station 1740, for weighting containers. For example, a scale station 1740 can be equipped with one or more scales to determine the weight of containers. An example of a scale includes a digital precision balance having a suitable accuracy. Weight determination may be useful to validate that the quantity of ingredients added has been dispensed according to a desired computer-readable script, or to determine the counterbalance weight needed when mixing with a planetary mixer that requires counterbalancing, for example.

In some embodiments, the automated compounding system 10 can include one or more labeling stations 1730 for labeling containers. During operation, labels could be applied to the containers. A label could associate a container with a specific compounded composition. Labels could be applied before, while and/or after making a compounded composition. Although labels could include material that is glued or otherwise attached to a container, this might not always be the case. For example, a label could be formed on or into a container or be printed directly onto the surface of a container. In general, markings could be applied to any of various types of containers and/or packages. In some embodiments, marking involves printing or otherwise producing labels and affixing labels to containers and/or packages. In other embodiments, marking could also or instead involve printing or otherwise forming markings directly on containers and/or packages. As such, features disclosed herein in the context of labels or labelling could be applied more generally to other types of marking. A label could include a written description of the container and/or the product in the container. A label could also or instead include a unique identifier that distinguishes a container from other containers. Examples of unique identifiers include letters, numbers, symbols, machine-readable code, and combinations thereof. The unique identifier could encode a description of a container and/or a product in the container. Unique identifiers on labels could include machine-readable code. For example, a barcode could be a matrix barcode or two-dimensional barcode, such as a Quick Response (QR) code. In some embodiments, a QR code could be used to navigate to a website hosted by the ingredient manufacturer/supplier, and the website could provide information related to the compounded composition. Machine-readable codes could also or instead be carried by “smart” labels such as radio frequency identification (RFID) chips or tags. An RFID chip could be integrated into a container or label, for example, and encode a unique identifier and/or information related to the container and/or its contents. Machine-readable codes that encode information represent one illustrative example of how information could be conveyed in markings on a label, container, or package. The information itself could be included in markings, for example. These examples, and/or other types of encoding or marking, could be used to convey any of various types of information.

The following is a non-exhaustive list of information types, any one or more of which could be included in a description of a container and/or a product in a container: batch number; lot number; compounding entity name, telephone number and/or email address; customer name, telephone number and/or email address; shipping information; Global Trade Item Number (GTIN); product name; product type; product composition; unit or case number; processing date(s); packaging date(s); safety information; regulatory information; expiration date (beyond use date—“BUD”); product/container weight; product/container volume; unit size; and the like.

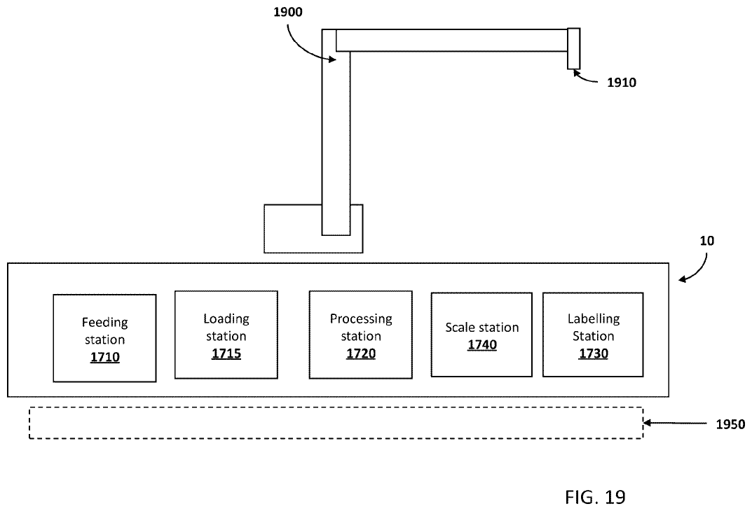

In some embodiments, the automated compounding system 10 may include one or more displacement means to displace containers within the automated compounding system 10. For instance, and with reference to FIG. 19, the displacement means can include one or more industrial robot 1900. Optionally, the displacement means may further include one or more conveyors 1950. For example, the one or more industrial robot 1900 can be equipped with an arm, i.e., a robotic arm or robotic manipulator. For example, the robotic arm can have an end of arm tool (EOAT) 1910 designed to manipulate containers having one or more pre-determined sizes and shapes. In most embodiments, the one or more industrial robot 1900 can be configured to pick (grip) a single container at a time although, in other embodiments, the one or more industrial robot 1900 may be configured to pick (grip) more than one container at a time. In some embodiments, the industrial robot 1900 may include a vacuum device or suction device to assist in picking the container(s). In some embodiments, the industrial robot 1900 can be equipped with a vacuum switch and can detect if a product is dropped between a pick and displacement operation, for example.

In some embodiments, the automated compounding system 10 may include a programmable logic controller (PLC), one or more barcode readers, and one or more vision systems. For example, the industrial robot can be a multiple-axis industrial robot, such as a 6-axis industrial robot. For example, a suitable multiple-axis industrial robot can be the IRB 1300-7/1.4 (ABB Ltd, Switzerland). For example, a suitable PLC can be the Compact GuardLogix 5069-L310ERMS2 (Allen-Bradley, United States). For example, a suitable barcode reader can be a Keyence SR-1000.

In some embodiments, the automated compounding system 10 may include a vision system comprising one or more image-capturing means (e.g., cameras) to capture the position of a container and transmit the images via a data line to an image-processing unit of the controller (described later), which evaluates these images. In some embodiments, the controller can control the displacement of the container by means of motors and can evaluate the selection, securing or release of the container based on the evaluation results derived from the image-processing unit. As will be apparent to the reader, however, other known means for capturing container location can be used, such as lasers. One or more image-capturing means can be installed at a specific location in the automated compounding system 10 and can provide real time digital images of the stage of the preparation process carried out at that location/station. An additional advantage of the visual control is a complete image-based documentation of the compounding process. This documentation can be kept in the automated compounding system 10 archive for future use, if needed. In some embodiments, the vision system is configured for detecting a container, sending position and orientation data to the industrial robot 1900 to pick the container. For example, the vision system may acquire and provide information from the container state and position, such as “at least one pickable container”, “container detected, but path blocked by unrecognized object”, “no container recognized”, “empty”, etc.

While specific functions are described above with respect to discrete stations, the reader will readily understand that one discrete station may combine two or more of such functions depending on specific examples of implementation, without departing from the present disclosure.

In operation, and with reference to FIG. 17, a controller 1700 interfaces with and controls the discrete stations of the system 10 through wireless or wired connections. In response to instructions received from the controller 1700, the plurality of discrete stations perform one or more process steps required for manufacturing the compounded composition. In response to instructions received from the controller 1700, the industrial robot 1900 is configured to displace one or more containers, for example from one discrete station to another discrete station, or bring a container to the processing apparatus, or place the container in the processing apparatus, or pick the container to remove same from the processing apparatus, or any combinations thereof.

In some embodiments, the automated compounding system 10 can have a modular architecture in the sense that the system can be designed to allow swapping components at one or more of the discrete stations. Such architecture can provide a technical effect in terms of system maintenance, flexibility and upgrading. For example, the system can be upgraded multiple times during its lifetime with the addition/replacement of one or more discrete station to increase capacity, modify functionality, and the like.

### Controller

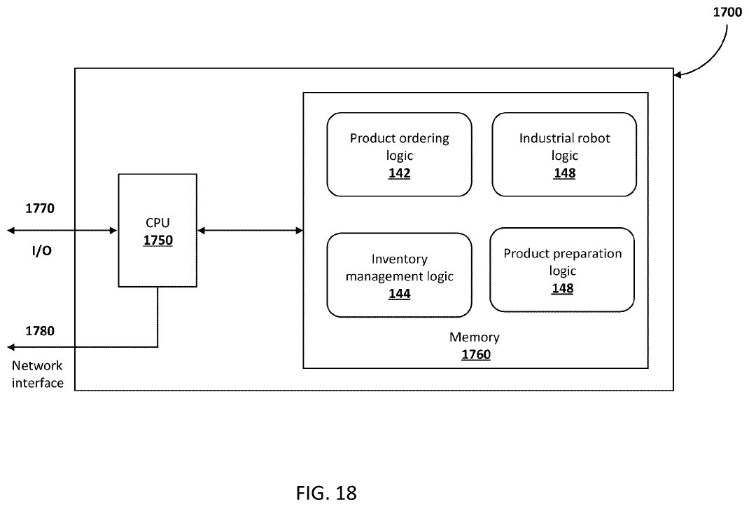

In a non-limiting implementation, and with further reference to FIG. 17 and FIG. 18, the controller 1700 is computer-based, including a data processor and a machine-readable storage encoded with software for execution by the data processor. The software defines logic, which determines how the system described herein operates to automate the compounded composition manufacturing process.

Specifically, the controller 1700 has an input/output (I/O) interface 1770, at least one data processor 1750 and a machine-readable storage, or memory, 1760. The readable storage, or memory, 1760 is encoded with software for execution by the data processor 1750. The input/output (I/O) interface 1770 allows the transfer of information to the outside world, such as external peripheral devices. The at least one data processor 1750 can be configured for communicating with a network interface 1780. The controller 1700 can include a general-purpose computer, a general-purpose processor (CPU), an application specific processor (ASIC), a circuit containing one or more processing components, a group of distributed processing components, or a group of distributed computers configured for processing, etc., configured to control the functionality of the automated compounding system 10 discussed herein.

While FIG. 18 illustrates the controller 1700 as being contained within a single enclosure, the reader will readily understand that a network of computers, for example a server arrangement, can implement the controller 1700. For example, one ore more stations may have one or more dedicated server(s), which are connected through wireless or wired connections with other servers of the automated system, together forming the controller 1700. Server, computer, and computing machine are meant in their broadest sense, and can include any electronic device with a processor including cellular telephones, smartphones, portable digital assistants, tablet devices, laptops, notebooks, and desktop computers. Examples of computer-readable media include, but are not limited to, CD-ROMs, flash drives, RAM chips, hard drives, EPROMs, etc.

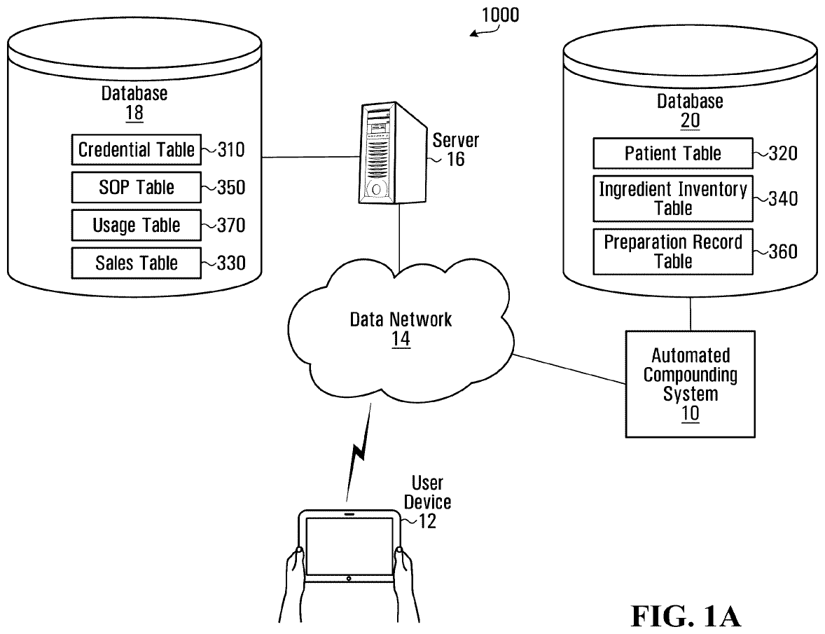

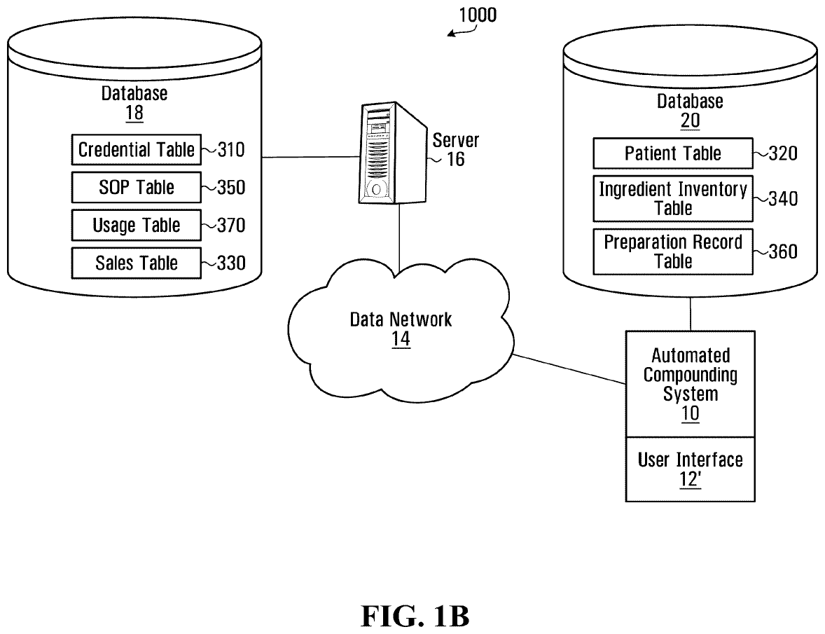

Optionally, the controller 1700 may interface and communicate with a user interface 12′ (as shown in FIG. 1B).

In some embodiments, the network interface 1780 enables communications between the controller 1700 and other devices or stations in the automated compounding system 10 or over data network 14. The structure of the network interface 1780 is implementation-dependent and may vary between embodiments that support different types of connections and/or communication protocols, for example. The network interface could enable communications over wired and/or wireless connections. In general, a network interface includes a physical interface such as a port, connector, or other component to interface with a communication medium, and a receiver and/or transmitter to process received signals and/or transmit signals for transmission.

In some embodiments, the machine-readable storage, or memory, 1760 could be, or include, one or more memory devices, such as one or more solid-state memory devices, and/or one or more memory devices that use movable or even removable storage media. The machine-readable storage, or memory, 1760 can be internal to the automated compounding system 10. A database 20 could be formatted or otherwise provided in the machine-readable storage, or memory, 1760 to store any or all information that is recorded by the automated compounding system 10. For example, the database 20 could store records, parameters, measurements and/or other information for recording and/or tracking by the automated compounding system 10. The database 20 may be part of the machine-readable storage, or memory, 1760 internal to the automated compounding system 10 or may be external to the automated compounding system 10 and connected directly thereto by a bus or over a data network (such as the Internet).

Typical of the wide variety of information that can be included in the machine-readable storage, or memory, 1760, for example in the form of a database 20 (which will be described later in this text) and used by the controller 1700 includes one or more of the following:
a) National Drug Code (NDC) numbers, barcodes, packaging dosage, physical properties, reconstitution, dilution dosages and dispensing instructions etc. of compounding ingredients. The information can be continuously or regularly updated, either automatically or manually, with information from sources such as the FDA, NIH, and industry.
b) Capacity, catalog numbers, National Drug Code (NDC) numbers, barcodes, or other identifying features of compounded composition. The information can be continuously or regularly updated, either automatically or manually, with information from the FDA, NIH and industry.
c) Capacity, catalog numbers, dimensions or other identifying features of containers and adaptors. The information can be continuously or regularly updated, either automatically or manually, from sources such as catalogues of medical products manufactures and distributers.
d) Images and video streams, both raw and processed taken by the image-capturing means (e.g., cameras).

It will be apparent to the reader that the controller 1700 can thus manage the compounding process and manage the data flow and the system database(s), such that the compounding process steps can be carried out automatically under guidance of the software and algorithms in the controller 1700 without, or substantially with minimal, human intervention.

Referring back to the data storage or memory, 1760, the software instructions provide a range of functions, which may include a product ordering logic block, or module 142, an inventory management logic, or module 144, an industrial robot logic, or module 148, and a product preparation logic, or module 150.

In some embodiments, the controller 1700 executes machine-readable code to implement the product ordering logic block, or module 142, which is configured to receiving a customer order. Such customer order may be generated online, for example. Typically, the product ordering block, or module, 142 can allow the customer to input a Prescription ID associated with a compounded composition. For example, inputting the Prescription ID may include scanning the Prescription ID (e.g., from a physical prescription order, from a label on a medication package, etc.) or inputting the Prescription ID (e.g., through an electronic file, user manual input, etc.). The product ordering logic block, or module, 142 can also allow the customer to communicate with the compounding facility for order management purposes, such as modifying the order if the order has not been fulfilled yet and canceling the order, among others. For example, the product ordering logic block, or module 142 can also allow the customer to receive shipping information.

In some embodiments, the controller 1700 executes machine-readable code to implement the product preparation logic, or module 150, which defines various actions performed at different stations, including the processing station for manufacturing the compounded composition.

In some embodiments, the controller 1700 executes machine-readable code to implement the industrial robot logic, or module, 148, which is designed to control and define the operations of the industrial robot 1900 to allow the industrial robot 1900 to pick and displace one or more containers from one station to another station, in the correct sequence.

In some embodiments, the controller 1700 executes machine-readable code to implement an inventory management logic 144, which effectively is a magazine inventory management system.

In some embodiments, the inventory management logic 144 can verify the inventory to determine if the system 10 includes sufficient containers and/or ingredients for manufacturing the compounded composition included in the customer order. For example, such determination step may be advantageous in the context of determining whether the system 10 is operational for fulfilling a specific customer order or a specific computer-readable script implementing a specific SOP script.

An SOP script can be a computer-readable digital or electronic file that encodes or specifies a particular “operating procedure” (sometimes referred to as a “standard operating procedure” or SOP), which may include an ordered set of process steps. The SOP script may also include the make and model of apparatus required for manufacturing a compounded composition. The SOP script may include the list of ingredients, their relative quantities, the total quantity/volume, as well order of addition into the container intended for placement into the processing apparatus.

In some embodiments, the inventory management logic 144 can manage containers inventory that are included in one or more container support devices, which are used during the automated compounding process.

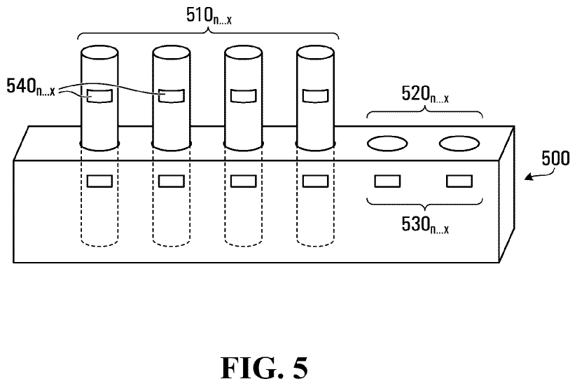

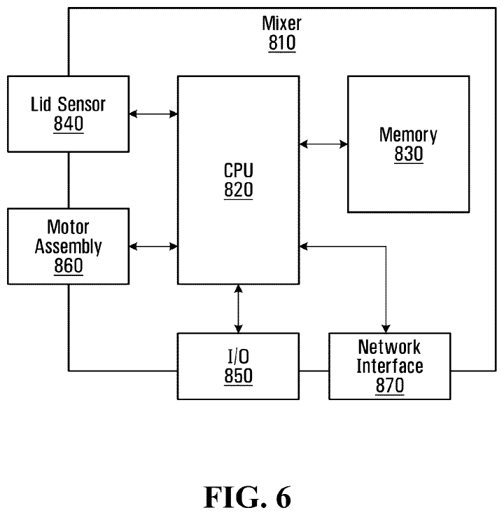

FIG. 5 illustrates a non-limiting example of a container support device in the form of magazine 500. The magazine 500 is configured for holding a plurality of containers 510n . . . x in respective container wells 520n . . . x. While the plurality of container wells 520n . . . x illustrated in FIG. 5 appear as apertures in the magazine 500, other forms of container wells can be envisioned and implemented in the magazine 500, without departing from the present disclosure.

Overall, the capacity to have one or more magazines 500 with possibly a large inventory of identical or different containers 510n . . . x constitutes a plethora of possible ingredients and/or containers available for manufacturing and storing compounded compositions, thus translating into more manufacturing flexibility. Indeed, being capable of manufacturing different compounded compositions without requiring frequent human intervention to provide the necessary ingredients or containers, represents a clear advantage over existing compounding systems and processes. The large number of possible different containers present at any given moment in the magazine 500 combined with the large spectrum of possible compounded compositions, however, represents a significant challenge to manage inventory. This challenge is advantageously addressed in the automated compounding system 10 with the herein described magazine inventory management system.

For example, the inventory management logic 144 can manage ingredient inventory by tracking usage of ingredients within a given container held in one or more magazines 500 and prompting an operator to perform a corrective action when the ingredient inventory in said container reaches a predetermined critical minimum threshold level and/or when the SOP calls for an ingredient quantity which cannot be met with the current ingredient inventory in said container. The corrective action can, for example, consist in replacing the ingredient container with a filled ingredient container or adding an ingredient container into the one or more magazines 500.

For example, the inventory management logic 144 can manage container inventory by tracking usage of containers held in one or more magazines 500 and prompting an operator of the compounding manufacturing facility to perform a corrective action when the container inventory reaches a predetermined critical minimum threshold level and/or when the SOP calls for a container or container quantity which cannot be met with the current container inventory. The corrective action can, for example, consist in replacing the containers present in the one or more magazines 500 or adding suitable containers into the one or more magazines 500.

A non-limiting example of implementation of the inventory management logic 144 with respect to ingredient inventory will now be described.

As shown in FIG. 18, the CPU 1750 is in communication with machine-readable storage, or memory, 1760 which may include a database, shown as database 20 in FIG. 1A. Database 20 includes various data that is used by the controller 1700 to provide the functionality discussed herein. In various embodiments, database 20 may include one or more of the following types of data:
magazine data representative of the magazine identifier,
data representative of containers to be manually placed in the wells of the magazine,
data representative of the respective wells within which the respective containers are to be placed,
data representative of the container items,
association data which associates the container identifier with the container,
alpha numeric data associated with the containers,
expiration dates associated with the ingredients,
manufacturer information associated with the ingredients, and
any other information which may be required for a particular application.

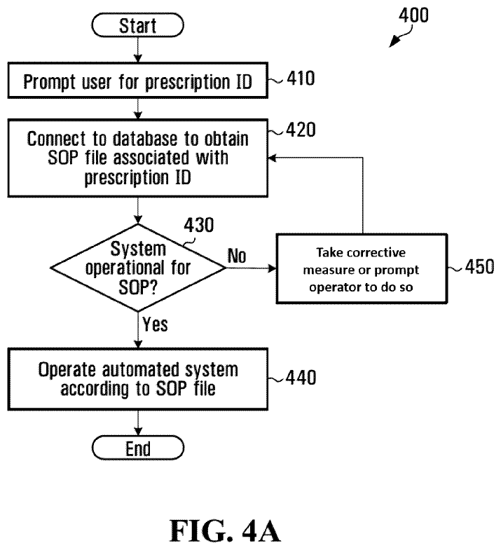

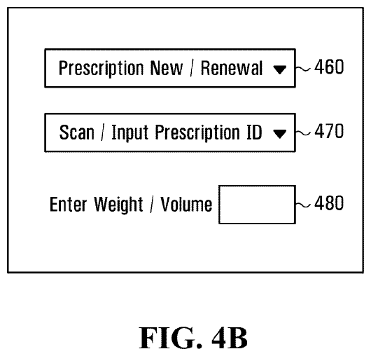

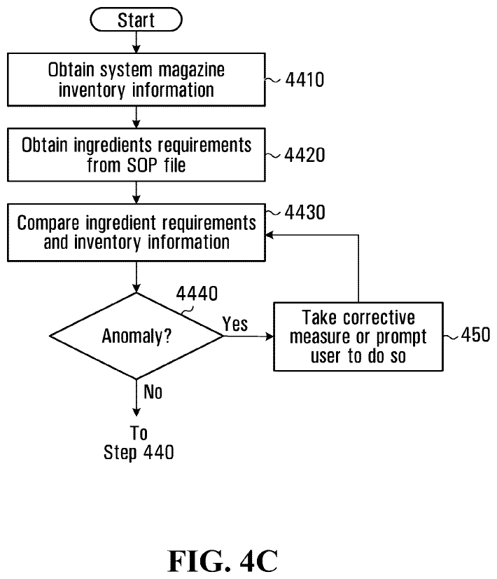

Referring to FIG. 4C, operation of the inventory management logic 144 with respect to ingredient inventory is shown according to an exemplary embodiment. At step 4410, the controller 1700 obtains magazine information associated with a magazine 500. Such information can include magazine identifier, container identifier, well identifier that holds a specific container, and the like.

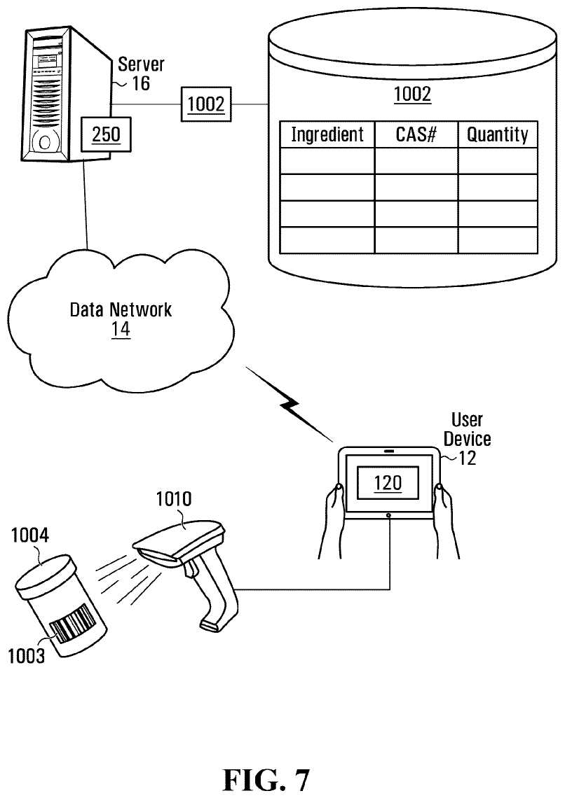

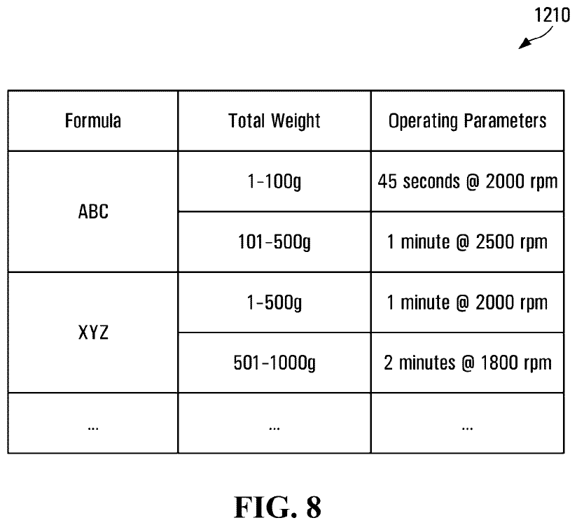

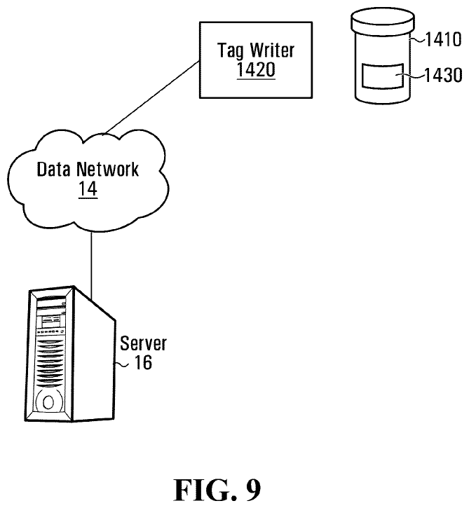

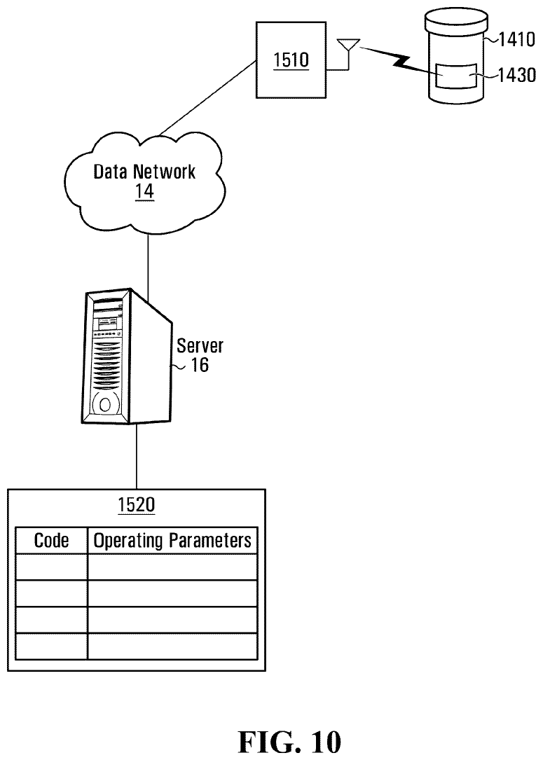

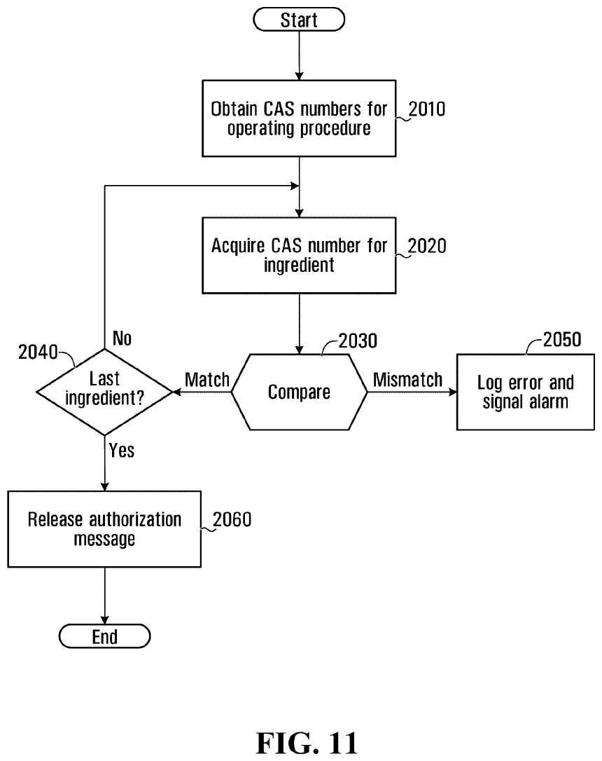

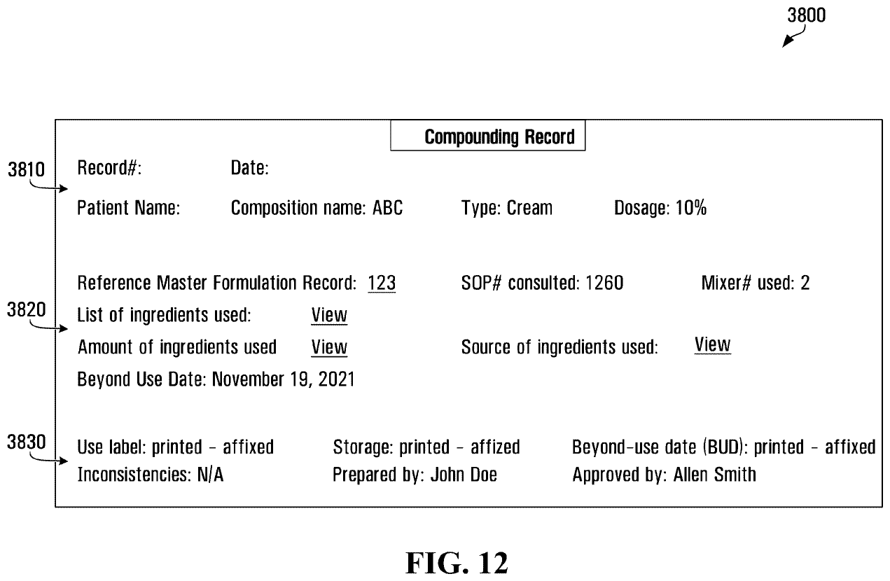

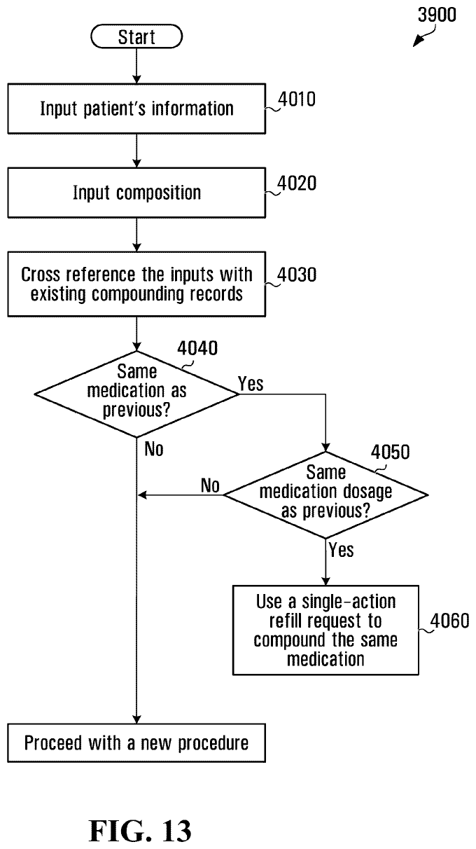

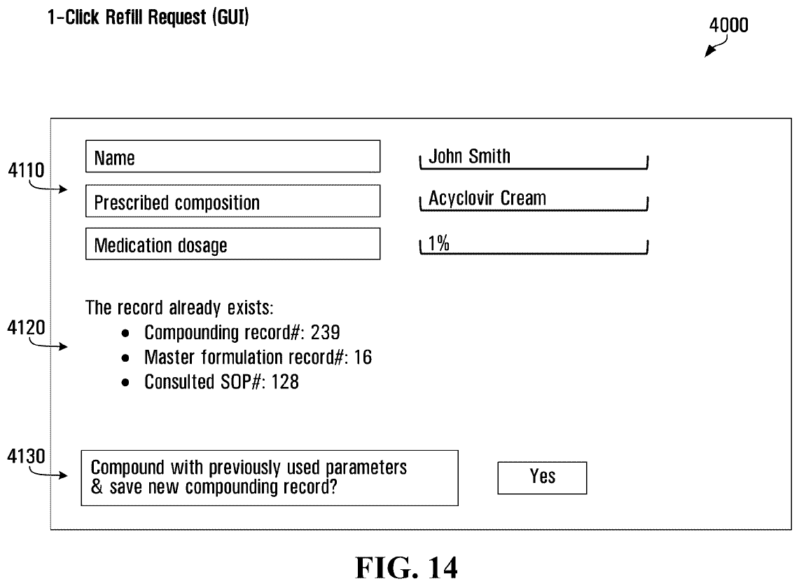

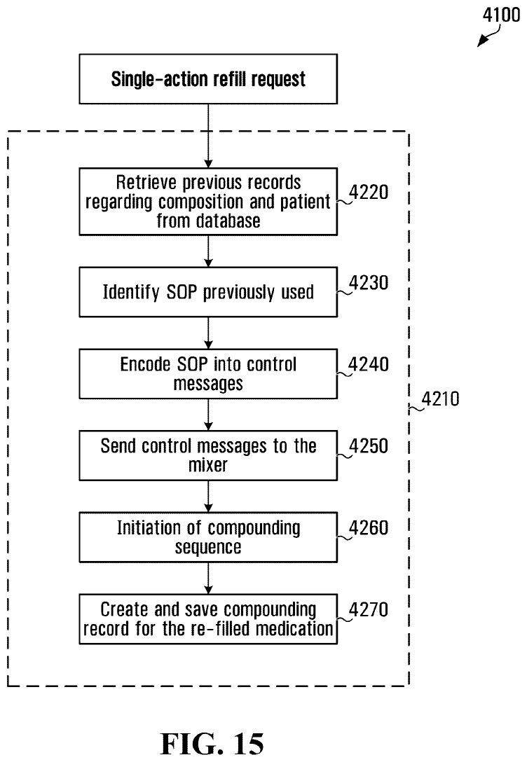

For example, the information relating to container identifier may be obtained by scanning one or more label of the plurality of labels 540n . . . x present on containers 510n . . . x, that are held in the magazine 500. Such scanning can be implemented with an automated data entry device 1010 (e.g., a scanner as shown in FIG. 7, which can be a barcode scanner, QR scanner or RFID reader). When the scanned containers are ingredient-containing containers, such information provides the control 1700 with an identity of the ingredients that are available in the magazine 500. The control 1700 can also derive ingredient the ingredient quantities that are available in respective containers. For example, the controller 1700 can derive such remaining ingredient quantity information from container-specific consumption tracking information stored in machine-readable storage, or memory, 1760.

At step 4420, the controller 1700 obtains the ingredient requirements for compounding a desired compounded composition based on the information contained in the computer-readable SOP script associated with the desired compounded composition. The ingredient requirements will dictate the identity and quantity of ingredient(s) required for implementing the compounding script contained in the computer-readable SOP script.

At step 4430, the controller 1700 compares the magazine information obtained at step 4410 with the ingredient requirements obtained at step 4420.

At step 4440, if an anomaly is detected, the controller 1700 will prompt an eventual operator (e.g., health care personnel, pharmacist, pharmacy technician, lab technician, etc.) to take corrective measure. For example, replacing one or more ingredient containers with suitable one or more ingredient containers or adding one or more suitable ingredient containers into the magazine 500. Additionally, the controller 1700 can ask the operator confirmation that the corrective measure has been completed.

It should be appreciated that some steps may be performed in a different order than what is illustrated in FIG. 4C, without departing from the present disclosure. A more detailed description of a magazine 500 will be discussed in the following section.

### Non-Limiting Example of Container Magazine

FIG. 5 illustrates a non-limiting example of a container support device in the form of magazine 500.

As discussed previously, the magazine 500 is configured for holding a plurality of containers 510n . . . x in respective container wells 520n . . . x. While the plurality of container wells 520n . . . x illustrated in FIG. 5 appear as apertures in the magazine 500, other forms of container wells can be envisioned and implemented in the magazine 500, without departing from the present disclosure.

Each container of the plurality of containers 510n . . . x may comprise a corresponding label from the plurality of labels 540n . . . x. Although labels could include material that is glued or otherwise attached to a container, this might not always be the case. For example, a label could be formed on or into a container or be printed directly onto the surface of a container, as will be further discussed later in this text. The information conveyed by the label from the plurality of labels 540n . . . x may be read by an automated data entry device 1010 (e.g., a scanner as shown in FIG. 7, which can be a barcode scanner, QR scanner or RFID reader) for identifying/validating the container located in each container mount of the plurality of container wells 520n . . . x.

For example, the data entry device 1010 may be used to acquire at least an identifier of an ingredient contained in an ingredient container from the plurality of containers 510n . . . x, in which case the label from the plurality of labels 540n . . . x may include the CAS number of the ingredient. This identifier may be used to validate that the correct ingredient container is present in the magazine 500 when assessing whether the system is operational for manufacturing a desired compounded composition. Such validation is part of the compounding process 400, which is further described elsewhere in this text. This acquisition of the at least one identifier of the ingredient contained in a container can be done by scanning a barcode scanner, QR scanner or RFID reader present on the label from the plurality of labels 540n . . . x. Such scanning requires that the magazine 500 is configured to allow the data entry device 1010 to access and scan the label from the plurality of labels 540n . . . x.

An example of the above principle is illustrated in FIG. 5, where a bottom portion of each container of the plurality of containers 510n . . . x is held within the body of the magazine 500 and is thus masked from the data entry device 1010, and where a top portion of each container of the plurality of containers 510n . . . x containing a label from the plurality of labels 540n . . . x is positioned above the top surface of the body of the magazine 500 such that the corresponding label is available for scanning by the data entry device 1010. Other examples of such principle can be envisioned and implemented in the magazine 500, without departing from the present disclosure.

In other non-limiting examples, the label from the plurality of labels 540n . . . x may also include information pertaining to a quantity of ingredient—this would be the case for example when the container of the plurality of containers 510n . . . x is a single-use container and therefore when the entire quantity of the ingredient within the container is to be used each time the label from the plurality of labels 540n . . . x is read by the data entry device 1000.

Optionally, the magazine 500 may comprise a plurality of labels 530n . . . x corresponding to the position of each container mount of the plurality of container wells 520n . . . x. The information conveyed by the label from the plurality of labels 530n . . . x may be read by the automated data entry device 1010 for identifying the position of a container mount of the plurality of container wells 520n . . . x. Such identification can be used for mapping the magazine 500 into the memory 1760 of the automated compounding system 10 for inventory management purposes of the various containers included in the magazine 500 and/or may be useful in the validation step 440 of the process 400.

### Customer Remote Ordering

In some embodiments, the automated compounding system 10 and/or the controller 1700 is communication-enabled or network-enabled to interface and communicate with a customer through user device 12 as shown in FIG. 1A.

For example, the user device 12 can be associated with a patient, a prescribing medical doctor, a health professional, a pharmacy technician, etc. For example, the user device 12 can include hardware and software components to implement a graphical user interface (GUI) that allows the customer to remotely request the manufacture of a compounded composition based on, for example, a medical prescription or other request. In other words, the GUI may allow ordering a compounded composition from a location remote from the automated compounding system 10.

In the present case, the term “customer” includes any one of a patient, a prescribing medical doctor, a health professional, a pharmacy technician, and the like.

In the present case, while the term “remote” is used, the reader will understand that the ordering could be performed inside the store, the pharmacy, or the hospital where the customer is located, but it is “remote” in the sense that the customer does not need to be in close vicinity of the automated compounding system 10. In some embodiment, it is possible for the customer to order a compounded composition remotely from the automated compounding system 10, for example while being at the medical doctor's office, while being in transit or at home, or it is possible for the customer to order a compounded composition while being onsite at the pharmacy that will compound the composition via a kiosk-style touch screen monitor self-ordering system, for example.

A non-limiting example of such remote ordering functionality will now be described with reference to FIG. 1A.

In some embodiments, the automated compounding system 10 and/or the controller 1700 is also communication-enabled or network-enabled to interface with the user device 12 as well as with one or more databases, thus operating within an automated compounding system environment 1000. In some embodiments, the automated compounding system environment 1000 includes the automated compounding system 10, the user device 12, a data network 14, a server 16, and a database 18. The server 16 can be in vicinity to the automated compounding system 10 or can be remotely located. As discussed previously, the automated compounding system 10 can further interface and communicate with database 20.

In some embodiments, the user device 12 is configured to communicate with the server 16 over the data network 14. In an embodiment, the user device 12 can be implemented as a mobile phone, a tablet, a desktop PC, or a console, to name a few non-limiting possibilities. The device 12 can include a memory, a processing entity (e.g., at least one processor), a screen and a network interface, amongst other standard components associated with the aforesaid implementations. The memory of the user device 12 may store computer-readable instructions that are executed by the processing entity of the user device 12 to cause the user device 12 to carry out certain processes.

In some embodiments, the data network 14 may be a public data network (such as the Internet) or a local area network. In an example of a non-limiting embodiment, the server 16 may be a World Wide Web server. In an embodiment, the database 18 may be part of a memory internal to the server 16 or may be external to the server 16 and connected thereto directly by a bus or over a data network (such as the internet). While the server 16 is illustrated in the figures as a single device, the reader will understand that in some embodiments, the server 16 may operate within a server arrangement including a plurality of servers, which may be present within a single physical location or may be decentralized at separate physical locations.

In some embodiments, the user device 12, along with the software associated to it, upon connecting to server 16 as well as accessing the database 18 and/or second database 20 may function as a functional hub through which a plurality of operations can be handled prior to, during and after compounding. A non-limiting list of such operations may include i) ingredients inventory management, ii) records management, iii) compounding process, iv) controlling auxiliary devices (e.g., barcode readers or scales), and v) accessing external databases (such as electronic health records), as further described in U.S. Patent Publication 2022/0,008,878, which is hereby incorporated by reference in its entirety.

Reference is now made to FIG. 4A and FIG. 4B, which show non-limiting flowcharts illustrative of a series of steps that may be executed by the controller 1700 to implement a graphical user interface (GUI) useful for initiating the compounding manufacturing process 400.

At step 410, the controller 1700 can cause the GUI to display a graphical element 460 (e.g., a dialog box list, or menu) that presents a set of prescription options, from which the customer is prompted to select an option. The set of prescription options may include a new prescription or a renewal of an existing prescription, for example. Further, the controller 1700 also can cause the GUI to display a graphical element 470 (e.g., a dialog box, list, or graphical menu) that presents a set of Prescription ID options, from which the customer is prompted to select an option. The selection can be between scanning the Prescription ID (e.g., from a physical prescription order, from a label on a medication package, etc.) and input the Prescription ID (e.g., through an electronic file, user manual input, etc.). Further, the controller 1700 also can cause the GUI to display a graphical element 480 that prompts the customer to enter a desired weight and/or volume for the compounded composition.

In some embodiments, the prescription ID may be a unique identifier associated with a compounded composition. For example, the unique identifier could encode a description of the compounded composition.

For example, in the case of a previously compounded composition, the prescription ID may be present on the label of the previously compounded composition. Unique identifiers on labels could include machine-readable code, such as a bar code or quick response (QR) code associated with a prescription. Machine-readable codes could also or instead be carried by “smart” labels such as radio frequency identification (RFID) chips or tags. An RFID chip could be integrated into a container or label, for example, and encode the unique identifier.

For example, in the case of a new compounded composition, the Prescription ID may be the name of the prescribed compounded composition (e.g., Topical cream (emulsion) containing 2% progesterone), or other information that is sufficient to clearly identify and distinguish a prescription from any other prescriptions.

It should be noted that the input of the desired weight/volume affects the control messages executable by the controller 1700. For instance, a lesser volume may require less mixing time. Accordingly, in response to the input from the customer specifying the desired weight/volume, the server 16 will customize the control messages executable by the controller 1700.

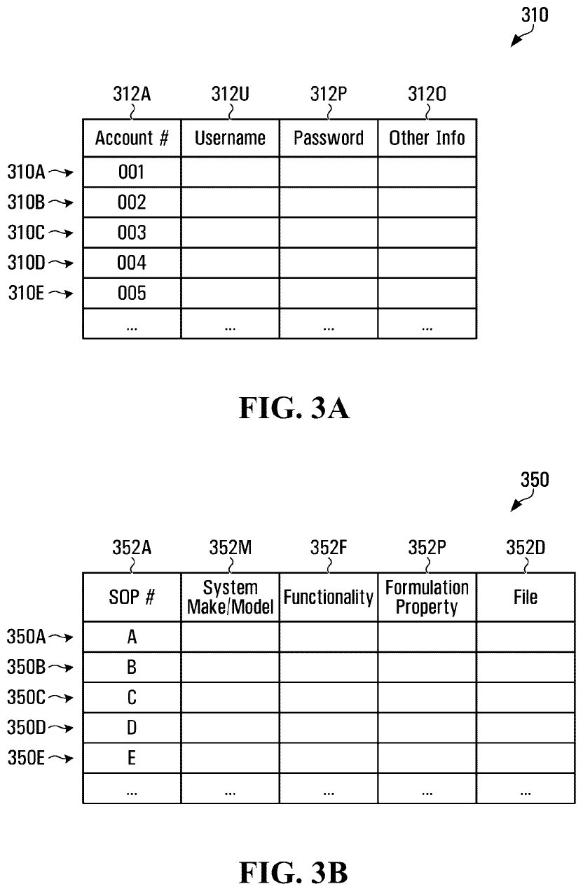

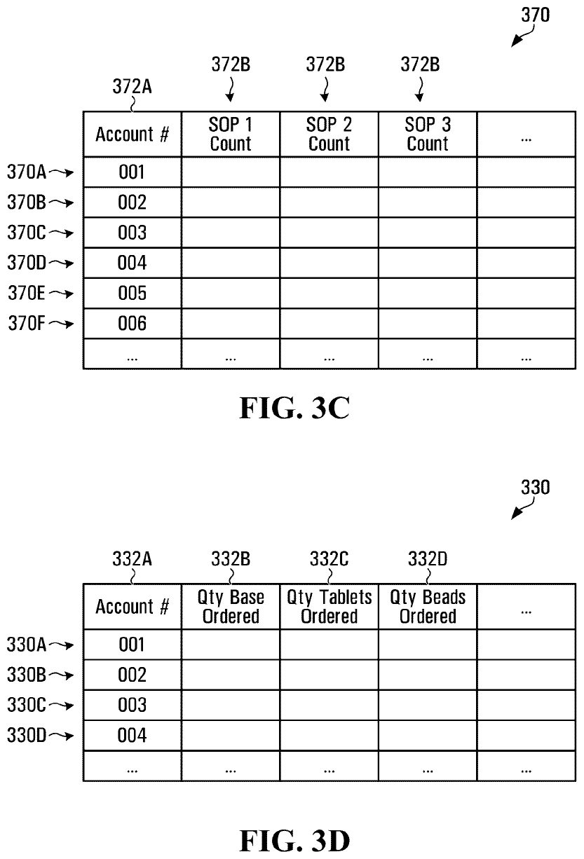

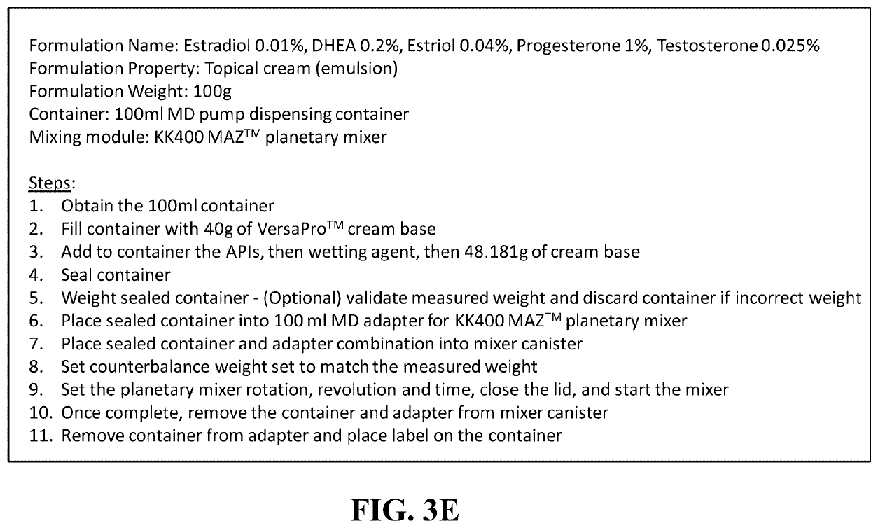

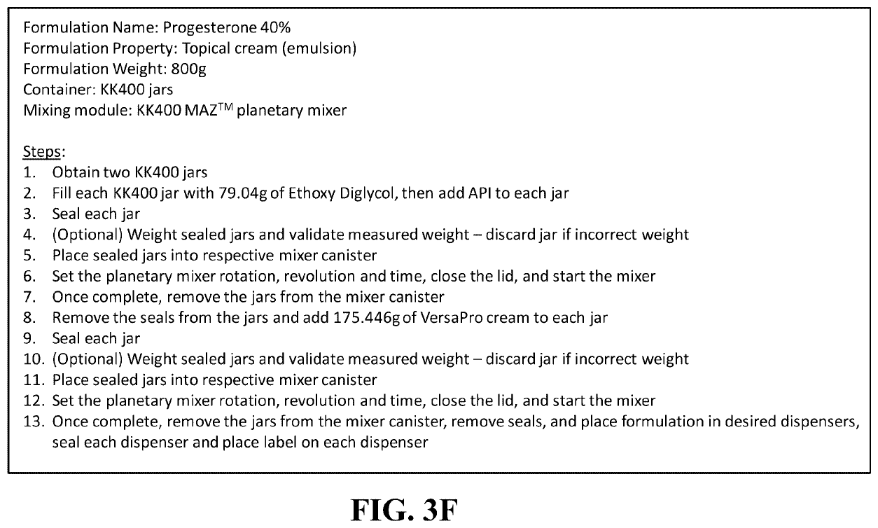

At this point in the process, the controller 1700 will have received the prescription ID and may have enough information to identify and obtain a computer-readable SOP script. This is attempted at step 420. In particular, the controller 1700 connects to database 20 and consults the SOP table 350 shown in FIG. 3B (described elsewhere in this text) to identify an appropriate one of the records 350A . . . 350E that matches the prescription ID, such that the corresponding computer-readable SOP script for the identified record is retrieved from memory, e.g., via the filename or pointer stored in the file field 352D for the identified record.

In some embodiments, the computer-readable SOP script can be received by the product preparation logic 148 of the controller 1700 over the data network 14. Encryption or password protection can be used to keep the contents of the computer-readable SOP script secure.

In some embodiments, the inventory management logic 144 then proceed to step 430 to assess whether the automated compounding system 10 is operational for the received computer-readable SOP script, as discussed elsewhere in this text. If the inventory management logic 144 determines that the automated compounding system 10 is not operational, then the controller 1700 may take the corrective measure or prompt an operator of the manufacturing compounding facility to do so, at step 450.

If the inventory management logic 144 determines that the automated compounding system 10 is operational, then the product preparation logic 148 proceeds to step 440 to operate the automated system according to the process steps of the computer-readable SOP script, which is associated with the Prescription ID.

The reader will readily understand that once the desired composition has been compounded as per instructions provided by the controller 1700, the GUI may include other interactive elements providing onsite pick up or delivery options, or payment charging options, for example. These are known in the art and will thus not be further described here.

Interface of the Device with External Database

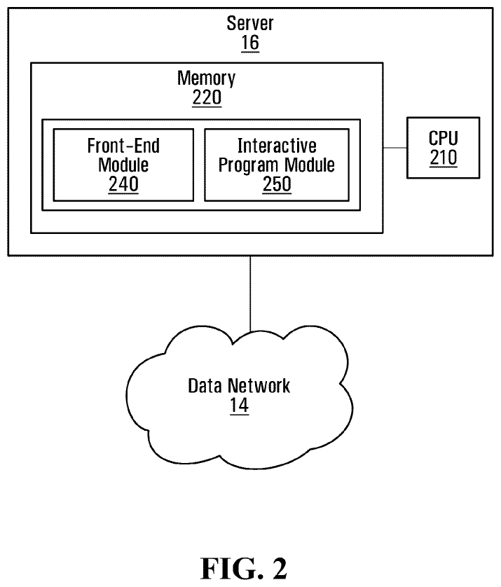

In some embodiments, and with reference to FIG. 2, the server 16 includes a processor (CPU) 210 and a memory 220 that stores computer-readable instructions executable by the processor 210.

In some embodiments, execution of the computer-readable instructions by the processor 210 causes the server 16 to implement a front-end module 240 and an interactive program module 250 (which may also be referred to as a “bot” or “wizard”). In some arrangements, the server 16 may be managed by, or property of, a manufacturer or distributor of composition compounding equipment and/or materials.

In some embodiments, the server 16 is accessible by the user device 12 via the data network 14. In other words, the user of the user device 12 may enter in communication with the server 16 by specifying a particular URL corresponding to the server 16, or by activating a software application (app) associated with the server 16. In response to being contacted at the URL (or via the app), the server 16 is configured to execute the front-end module 240. The front-end module 240 may cause an activatable link or icon corresponding to the interactive program module 250 to be presented on the screen of the user device 12. The front-end module 240 is configured to detect activation (e.g., clicking or selecting through the user device 12) of the activatable link or icon and, in response to such activation, may launch the interactive program module 250. Launching the interactive program module 250 may include executing computer-readable instructions which, when executed by the processor 210, cause the server 16 to present a graphical user interface to the user device 12 over the data network 14.

In some embodiments, the front-end module 240 may perform an account verification process before allowing the link or icon to be activated. To this end, reference is made to FIG. 3A, in which are shown possible contents of the first database 18 conceptualized as a table 310 of records 310A . . . 310E, hereinafter referred to as a “credentials table”. Each of the records 310A . . . 310E in the credentials table 310 includes a plurality of entries, in each of an account #field 312A, a username field 312U, a password field 312P and possibly other fields (e.g., other information field 3120). For a given one of the records 310A . . . 310E, the entry in the account #field 312A is information corresponding to a particular account holder associated with that record (e.g., it could be a unique account number or customer name or legal entity name). The entry in the username field 312U and password field 312P represents credential information that may be set up by the account holder during a prior “account setup” phase. The entry in the other info field 3120 could be related to other information associated with the account holder, such as make and model of a mixer known to have been purchased by the account holder, the date of purchase, the software version and servicing information, to name a few non-limiting possibilities. Of course, those skilled in the art will appreciate that the reference to a “table” being used for account information is merely a conceptualization of an otherwise tangible and non-transitory medium for storing such information. In this regard, the first database 18 may implement any suitable organizational format or data structure in lieu of the credentials table 310.

In one possible variant, the connection between the server 16 and the user device 12 may be made via the automated compounding system 10. To elaborate, the controller 1700 can communicate with the server 16 and with the user device 12. The communication with either entity may be wireless or wireline. Alternatively, the device 12 may be physically integrated into the automated compounding system 10 (in the form of user interface 12′, as shown in FIG. 1B) and such as a display screen through which the customer interacts directly with the automated compounding system 10 and with the server 16. In this instance, the automated compounding system 10 may automatically supply to the server 16 the identification information such as the model number, software versions, enabled functions, such as to let the server 16 know what are the capabilities of the automated compounding system 10 and the mixer 810 identity. The user credentials can be input via the user device 12, irrespective of the type of implementation, separate from the automated compounding system 10 or integrated therewith.

Returning now to FIG. 2, as part of the account verification process, the front-end module 240 may be configured to request credentials (e.g., a username and/or password) from the user device 12. The front-end module 240 verifies these credentials against the information stored in the credentials table 310 of the first database 18 and requires a match before the interactive program module 250 is launched and presented to the customer via the user device 12. Once the correct credentials corresponding to a particular account holder are entered, the front-end module 240 from now on assumes that all interactions with the customer are on behalf of the rightful account holder.

Note that the account verification process may also include a verification of the identity of the automated compounding system 10 and/or the controller 1700 to ensure it is an authorized network device. For instance, the server 16 may be configured such that it provides services only to authorized systems and before proceeding with delivering services, the process will verify if the automated compounding system 10 and/or the controller 1700 is on the authorized list. The verification may include receiving from the automated compounding system 10 and/or the controller 1700, in addition to the model number and other characterizing information a unique identifier that distinguishes the system from other systems of the same model. For instance, the unique identifier may be a serial number that is encoded in the and/or the controller 1700 memory in tamper proof fashion.

In the present embodiment, according to one of its functionalities, the interactive program module 250 is configured to obtain and transmit the computer-readable SOP script to the controller 1700.

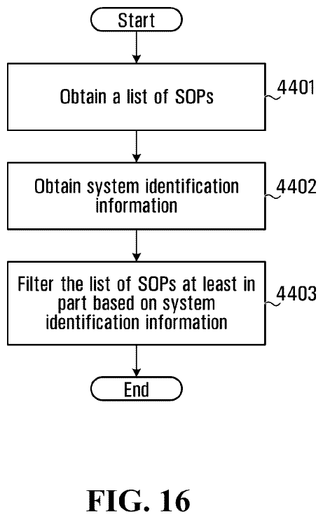

As it will be discussed later, the computer-readable SOP script may be filtered based on the capabilities of the automated compounding system 10. For instance, the database 18 has a list of computer-readable SOP scripts, some of which may be compatible only with certain mixers and/or with specific systems. As illustrated in FIG. 16, to avoid sending the user wrong computer-readable SOP script information, the server 16 will first obtain a list of available computer-readable SOP scripts at step 4401 and subsequently obtains automated compounding system 10 identification information at step 4402. The server 16 after obtaining the automated compounding system 10 identification information may filter the computer-readable SOP scripts at step 4403 based on the automated compounding system 10 identification information such that the information transmitted will work with the automated compounding system 10. It will be appreciated that the order of steps 4401 and 4402 may be reversed. For example, the server 16 may obtain automated compounding system 10 identification information first and then obtain all available computer-readable SOP scripts which will subsequently become filtered based on the automated compounding system 10 identification information. This filtering is useful as the server 16 also sends programming information (i.e., operational scripts), e.g., specifying the mixer settings, the list of ingredients, the movement flow, and the like, to implement a particular computer-readable SOP script. The automated compounding system 10 programming information can be sent as machine parameters directly to the memory of the and/or the controller 1700. The controller 1700 is thereby automatically programmed as required for the computer-readable SOP script.

In some embodiments, the first database 18 may be used for storing the information relevant to multiple operating procedures. In this regard, and with reference now to FIG. 3B, there is shown possible contents of the first database 18, conceptualized as a table 350 of records 350A . . . 350E in memory, hereinafter referred to as an “SOP table”. Each of the records 350A . . . 350E is associated with a respective operating procedure and includes a plurality of entries in a plurality of fields. The fields may include an SOP #field 352A, a mixer make/model field 352M, a functionality field 352F, a formulation property field 352P and a file field 352D, as possible examples. These fields are now described in further detail.

In some embodiments, the SOP #field 352A for the record associated with a given operating procedure stores a unique identifier of the given operating procedure. The “functionality” field 352F may specify information regarding a specific use of the automated compounding system 10. Non-limiting examples of functionality are varied and can include one or more of (i) particle size reduction (micronization/grinding); (ii) melting; (iii) mixing of powders; (iv) mixing of creams/gels/topicals and (v) de-aerating (degassing), for example. The “formulation property” field 352P may specify information regarding the specific delivery form, dosage or other properties of the formulation that is to be produced by the automated compounding system 10. Non-limiting examples of formulation property are varied, and in some cases, it is not an independent choice but rather depends on the automated compounding system functionality. In some embodiments, formulation properties are selectable, and a set of selectable formulation properties may be different for at least two different automated compounding system functionalities, as discussed earlier.

For example, in a non-limiting embodiment, one can have the following association between automated compounding system functionality and formulation property:

TABLE 1

| System functionality | Formulation property |
|---|---|
| Particle size reduction | Lidocaine powder; |
| | Crystallized and large particle powders |
| Tablets | Enteric coated; |
| | Non-coated |
| Homogenizing powders | Progesterone capsules; |
| | Bi-estrogen capsules without progesterone; |
| | Bi-estrogen capsules with progesterone; |
| | Non-HRT powders |
| Mixing cream/gel/topical | HRT Cream containing 10% or less |
| compounds | active ingredients; |
| | HRT Cream containing 10% or more |
| | active ingredients |

Another example of the “system functionality” could be whether an adapter is to be used in the mixing process, such adapter being configured to hold the final dispensing container from which the prepared formulation is to be dispensed. This contrasts with the situation where, once mixed in a mixing container, the mixed formulation is to be transferred from the mixing container into a dispensing container. As such, if the system functionality is selected to be “adapter mixing”, there may be an implicit assumption that the intended use of the system is for mixing a cream/gel/topical compound in a dispensing container. This may lead to additional granularity (which can be referred to as a sub-functionality) when selecting the “adapter mixing” functionality.

For example, the following table lists possible system functionalities:

TABLE 2

| System functionality | System sub-functionality |
|---|---|
| Adapter mixing | Pump adapter |
| Adapter mixing | 15 ml-50 ml Unguator jar adapter |
| Adapter mixing | 100 ml Unguator jar adapter; |
| Adapter mixing | 100 ml Samix jar adapter |
| Adapter mixing | Universal adapter |

In some embodiments, the “system make/model” field 352M for the record associated with a given operating procedure is optional and may identify the manufacturer, model, and modules of automated compounding system for which the given operating procedure has been designed. This is to accommodate differences in the various modules that may be included in the system. For example, a given system may have a mixing module that includes a planetary mixer that requires calibration of RPM, counterbalance (if applicable), mixing time and order of steps which may differ from one manufacturer, model, or size of mixer to another, even where the functionality and the formulation properties remain the same.

Finally, the “file” field 352D for the record associated with a given operating procedure includes a name of, or a pointer to, a computer-readable SOP script, namely a digital or electronic file that encodes or specifies the given operating procedure. The way in which a computer-readable SOP script encodes or specifies a given operating procedure is not particularly limiting. By way of non-limiting example, the computer-readable SOP script could be a text file that specifies the ordered set of process steps forming the given operating procedure or machine-readable code (script) that provides instructions to the controller 1700 without or with minimal intervention from an user.

As mentioned above, the operating procedure encoded or specified by the computer-readable SOP script may include a sequence of ordered process steps. Some of these process steps for operating the automated compounding system may involve the setting of “operating parameters” for controlling operation at one or more stations of the automated compounding system 10. The operating parameters, as discussed earlier, can in some cases be provided to the controller 1700 using an input/output interface of the and/or the controller 1700 (such as a screen display with key/button input). For example, on reception of a computer-readable SOP script, the product preparation logic 148 implements the computer-readable SOP script by sending instructions to components of the automated compounding system 10 such as the industrial robot 1900, to automate manufacturing steps.

For example, in response to instructions received from the controller 1700, more particularly of the product preparation logic 148, the industrial robot 1900 will grip a first container from the magazine of empty containers in the feeding station 1710, and place the first container on a scale in the loading station 1715. The industrial robot 1900 will then grip a first ingredient container from the feeding station 1710 containing sufficient amounts of a first ingredient, and a scanner (e.g., barcode scanner, QR scanner or RFID reader) scans the label on the first ingredient container to validate that the ingredient contained therein corresponds to the first ingredient in the SOP script. Once validated, an amount of the first ingredient is poured in the first container, using suitable means such as vacuum assisted pipettes or syringes, etc. The industrial robot 1900 will then place the first ingredient container back into its initial location and will grip a second ingredient container from the feeding station 1710 containing sufficient amounts of a second ingredient. The scanner (e.g., barcode scanner, QR scanner or RFID reader) then scans the label on the second ingredient container to validate that the ingredient contained therein corresponds to the second ingredient in the SOP script. Once validated, an amount of the second ingredient is poured into the first container, again using suitable means such as vacuum assisted pipettes or syringes, etc. The industrial robot 1900 will then place the second ingredient container back into its initial location and will grip the first container and place same in the processing apparatus called out in the computer-readable SOP script. The controller 1700 will then configure the processing apparatus for processing the ingredients based on parameters specified in the computer-readable SOP script, and start the operation of the apparatus. When the operation of the apparatus ends, the controller 1700 then optionally instructs the industrial robot 1900 to pick the container containing the processed ingredients and place same on a scale in the scale station 1740 to validate the container weight against the expected weight. If assessing final weight is required in the SOP script, once validated, the industrial robot 1900 will pick the container containing the processed ingredients and displace same to the labelling station 1730, where a label is affixed to the container containing the processed ingredients for shipping to the customer or for sale to the customer.

Examples of operating parameters that may be specified in each process step of a given operating procedure may include, without limitation, one or more of: mixing speeds (e.g., rotation speed (in rpm) and/or revolution speed (in rpm) of a planetary mixer); order of addition of ingredients; displacement of containers (e.g., move from one station to another); handling of containers (e.g., seal the container, remove seal); affixing label on container; and the like. For example, the operating parameters may provide instructions that when executed by the controller 1700 cause the automated compounding system 10 to displace the container from a first station to a second station.

With reference to FIG. 3E, there is shown a generalized non-limiting example of contents of a computer-readable SOP script for a first non-limiting operating procedure. In this example, the functionality is “Mixing topical cream (emulsion)” and where the formulation property is “HRT Cream Containing 10% or Less of Active Ingredients”. This first operating procedure includes 11 process steps, including container displacement instructions.

With reference to FIG. 3F, there is shown a generalized non-limiting example of contents of a computer-readable SOP script for a second non-limiting operating procedure. In this example, the mixer functionality is “particle size reduction” and where the formulation property is “HRT Cream Containing 10% or more of Active Ingredients”. This second operating procedure includes 13 process steps, including container displacement instructions for the industrial robot 1900.

Of course, those skilled in the art will appreciate that the reference to a “table” being used for storing records (including links/pointers to computer-readable SOP scripts) is merely a conceptualization of an otherwise tangible and non-transitory medium for storing such information. In this regard, the first database 18 may implement any suitable organizational format or data structure in lieu of the SOP table 350.

In an embodiment, the controller 1700 may be configured to track the frequency with which the computer-readable SOP scripts are consulted or retrieved by each account holder. In some embodiments, an artificial intelligence (AI) module, executed by the controller 1700 or by another computing device may be used to learn which types and/or quantities of compounded compositions tend to be ordered together. In so doing, the AI module may be able to recommend keeping larger inventory amounts of specific ingredients required for such compounded compositions that tend to be ordered together. The AI module may thus be able to recommend acquiring different ingredients or larger quantities of specific ingredients. The AI module may also take into account various factors, such as season (e.g., spring, summer, fall, and winter), customer geography, discounts or sales promotions or other market trends or factors.

In some embodiments, and with reference to FIG. 3C, there is shown a table 370 of records 370A . . . 370F, hereinafter referred to as a “usage table”. Each of the records 370A . . . 3F0D includes a plurality of entries, in each of an account #field 372A and a plurality of SOP count fields 372B. For a given one of the records 370A . . . 370F, the entry in the account #field 372A is information corresponding to a particular account holder associated with that record, as previously described (e.g., it could be a unique account number or customer name or legal entity name). As for the SOP count fields 372B, each of these fields may pertain to a different computer-readable SOP script that was requested by the account holder in question. Of course, those skilled in the art will appreciate that the reference to a table is merely a conceptualization, as other organizational formats or data structures may be suitable and may thus be employed instead of a table. It will be appreciated that the usage table 370 may further store the times at which the various computer-readable SOP scripts were requested by the various account holders.

In cases where the entity that maintains the server 16 is not only a supplier of computer-readable SOP scripts for the controller 1700 but is also a supplier of ingredients used in mixing, additional information pertaining to the account holders is available to this entity. This additional information may also be stored in the first database 18. This is conceptually illustrated in FIG. 3D, where there is shown a table 330 of records 330A . . . 330D, hereinafter referred to as a “sales table”. Each of the records 330A . . . 330D in the sales table 330 includes a plurality of fields, including an account #field 332A and a plurality of purchased ingredient quantity fields 332B, 332C, 332C, 332E. For a given one of the records 330A . . . 330D, the entry in the account #field 332A is information corresponding to a particular account holder associated with that record, as previously described (e.g., it could be a unique account number or customer name or legal entity name). As for the purchased ingredient quantity fields 332B, 332C, 332D, 332E, each of these fields may pertain to a different item that has been purchased by the account holder in question.

The granularity with which the purchased ingredients are designated by the purchased ingredient quantity fields 332B, 332C, 332D, 332E depend on the record keeping practices of the supplier. At a very coarse level, the purchased ingredients designated by the purchased ingredient quantity fields 332B, 332C, 332D, 332E may be categorized as base, chemical, color, flavor, oil, excipient powder, tablets and capsule, for example. At a finer level of granularity, each of the aforementioned categories could be expanded, in some cases down to the molecular level (e.g., Acesulfame Potassium, Acetaminophen, Acetazolamide, etc.). As such, the number of purchased ingredient quantity fields 332B, 332C, 332D, 332E may be very large when it is desired to keep detailed records on the purchasing history of account holders. It will be appreciated that the sales table 330 may further store the purchasing timeline of the various items purchased by the various account holders.

It will be appreciated that the credential table 310 and the sales table 330 could be merged, as each includes a respective account #field, which could refer to the same account holder.

### Ingredient Validation

In some embodiments, the manufacture process includes keeping track of which ingredients are added to a container. In some embodiments, the controller may be configured to validate the collected data before authorizing the processing apparatus to be started.

For example, it may be possible to explicitly track the ingredients being added to a container so as to gain improved quality control and/or business insight. In this regard, it is noted that many ingredients approved for use in a pharmacy setting are associated with a unique CAS number (a unique numerical identifier assigned by the Chemical Abstracts Service (CAS) to every chemical substance described in the open scientific literature). CAS numbers can appear on the bottles and containers of the various ingredients to be used in creating the formulation of choice. The CAS numbers may even be encoded in a bar code or QR (quick response) code on the various ingredient containers.

In some embodiments, the system described herein may include one or more code scanner (including, but not limited to, a barcode, QR, RFID scanner), connected to the controller 1700. The scanner may capture a code associated with an ingredient container, where the code includes information associated with the CAS number of an ingredient contained in a container included in the autonomous system described here. The controller 1700 may execute a validation process, based on CAS information included in the SOP script associated with a Prescription ID, or included in a database to which connects the controller 1700. Optionally, the controller 1700 may be configured to disable the processing apparatus from starting the processing steps until the validation process has successfully completed.

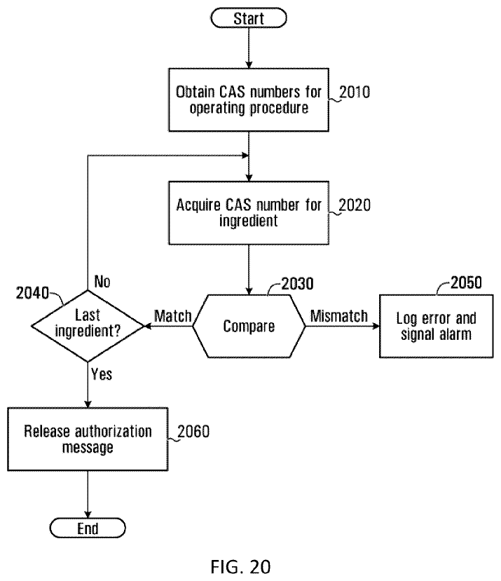

In some embodiments, and with reference to FIG. 20, an initialization step 2010 may be provided, whereby the controller 1700 contacts the database to obtain the CAS numbers of the ingredients associated with a desired SOP script or Prescription ID. At step 2020, according to the validation process, ingredient data is acquired by the one or more code scanner. This can be a CAS number or image data that encodes a CAS numbers. At step 2030, the validation process compares the acquired CAS number to the CAS numbers of the ingredients associated with the SOP script or Prescription ID. At this point, the validation process carries out an action that depends on the result of the comparing. For example, if there is a match, then the next step could be step 2040. If there is a mismatch, i.e., one of the acquired CAS numbers does not match any of the CAS numbers for the ingredients listed in the SOP script or Prescription ID, then the validation process may, at step 2050, log the error and also to signal an alarm in real-time, in the form of a message, audible or visual cue. This can immediately alert an operator that there is a problem, potentially resulting in less wastage of time and material resources. An optional step (not shown) may check to determine whether the CAS number has been duplicately scanned and, if so, to issue an alarm.

At step 2040, the validation process determines whether the acquired CAS number corresponds to the last ingredient that needed to be scanned for the selected SOP script of Prescription ID. If so, the controller 1700 may, at step 2060, enable the processing apparatus to authorize it to commence processing. This can be done by sending an authorization instruction message to the processing apparatus. Until the authorization instruction message is received by the processing apparatus, further processing may be blocked (i.e., the functionalities of the processing apparatus may be disabled).

### Ingredients for Manufacturing Compounded Compositions

In some embodiments, the automated system described herein may be used for manufacturing compounded compositions, such as compounded pharmaceutical, cannabis, or cosmetic compositions.

In some embodiments, ingredients that may be used for manufacturing such compounded compositions include one or more active pharmaceutical ingredient (API), cannabis-derived ingredient, or cosmetic ingredient, one or more excipient, solvent or diluent, and any combinations thereof.

In some embodiments, ingredients for manufacturing compounded cosmetic compositions may include active over the counter (OTC) ingredients or prescription pharmaceutical ingredients. Within the context of the present specification, OTC and prescription ingredients are encompassed by the expression API.

Examples of active pharmaceuticals ingredients (APIs) include, but are not limited to, antibiotics, analgesics, vaccines, anticonvulsants; antidiabetic agents, antifungal agents, antineoplastic agents, antiparkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hypothyroid, anti-hyperthyroid, antiasthmatics and vertigo agents.

In some embodiments, a pharmaceutically acceptable excipient, diluent or carrier may be a solid, semi-solid (more or less viscous fluid) or fluid (for example a cream or an emulsion). The person of skill will appreciate that pharmaceutically acceptable excipients, diluents or carriers are known in the art and may include, but without being limited thereto, anti-adherents such as magnesium stearate; binders, such as saccharides and their derivatives (sucrose, lactose, starches, cellulose or modified cellulose, sugar alcohols such as xylitol, sorbitol or maltitol), proteins such as gelatins, synthetic polymers such as polyvinylpyrrolidone (PVP) or polyethylene glycol (PEG); coloring dyes or fragrance; glidants such as fumed silica, talc, and magnesium carbonate; hydrophilic or hydrophobic lubricants such as talc or silica, and fats, e.g. vegetable stearin, magnesium stearate or stearic acid; preservatives such as antioxidant vitamins or synthetic preservatives like parabens; sorbents or other desiccant; vehicles that serve as a medium for conveying the active ingredient such as petrolatum, gum base gelatin, dimethyl sulfoxide and mineral oil.

In some embodiments, the pharmaceutically acceptable excipient, diluent or carrier may include a commercial product such as VersaPro™ Gel, HRT™ Cream, OleaBase™ Plasticized, PLO Gel Mediflo™, Oral Mix™, VersaPro™ cream, and the like, all from Medisca Pharmaceutique (Canada).

In some embodiments, the compounded compositions may be adapted for oral, rectal, vaginal, topical, urethral, ocular, or transdermal administration.

Other examples of implementations will become apparent to the reader in view of the teachings of the present description and as such, will not be further described here.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

All references cited throughout the specification are hereby incorporated by reference in their entirety for all purposes.

Reference throughout the specification to “some embodiments”, and so forth, means that a particular element (e.g., feature, structure, and/or characteristic) described in connection with the invention is included in at least one embodiment described herein, and may or may not be present in other embodiments. In addition, it is to be understood that the described inventive features may be combined in any suitable manner in the various embodiments.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

Although various embodiments of the disclosure have been described and illustrated, it will be apparent to those skilled in the art considering the present description that numerous modifications and variations can be made. The scope of the invention is defined more particularly in the appended claims.

1. An automated compounding system for manufacturing compounded compositions, the system comprising:
(a) a controller including a data processor for receiving via data communication over a data network a customer order, the customer order identifying a Prescription ID associated with a compounded composition,
(b) a plurality of discrete stations, including
(i) a processing station comprising a processing apparatus configured, in response to instructions received from the controller, to perform one or more process steps required for manufacturing the compounded composition, and
(ii) a plurality of containers,
(c) an industrial robot configured, in response to instructions received from the controller, to displace one or more containers of the plurality of containers from one discrete station to another discrete station.

2. The system of claim 1, wherein the system has a modular architecture.

3. The system of claim 1, wherein the controller implements an ordering logic configured to receive and process the customer order.

4. The system of claim 1, wherein the data processor communicates with a database to obtain a computer-readable standard operating procedure (SOP) script associated with the Prescription ID.

5. The system of claim 4, wherein the controller implements a product preparation logic configured to define a sequence of operations for manufacturing the compounded composition at least based on the SOP script.

6. The system of claim 1, wherein the controller implements an inventory management logic to determine inventory status of the containers.

7. The system of claim 6, wherein based on the determined inventory status, the controller determines whether the system is operational to fulfill the customer order.

8. The system of claim 1, wherein the plurality of discrete stations includes a feeding station equipped with one or more container support devices configured for holding the plurality of containers.

9. The system of claim 8, wherein the plurality of discrete stations includes a loading station for adding ingredients into a container from the plurality of containers.

10. The system of claim 9, wherein the industrial robot is configured, in response to instructions received from the controller, to sequentially displace a plurality of ingredient containers from the feeding station to the loading station.

11. The system of claim 9, wherein the industrial robot is configured, in response to instructions received from the controller, to pick the container containing the added ingredients and bring the container to the processing apparatus.

12. The system of claim 1, wherein the processing apparatus includes one or more planetary mixer(s).

13. The system of claim 11, wherein in response to instructions received from the controller, the processing apparatus is configured to subject ingredients in the container to at least one of mixing, grinding, de-aerating, melting, and any combination thereof.

14. The system of claim 1, wherein the plurality of discrete stations includes a scale station equipped with one or more scales to determine a weight of containers.

15. The system of claim 1, wherein the plurality of discrete stations includes a labeling station for labeling containers.

16. The system of claim 1, wherein the controller includes a machine-readable storage encoded with software for execution by the data processor.

17. The system of claim 1, wherein a server arrangement implements the controller.

18. A computer-implemented method of manufacturing a compounded composition, the method comprising:
(a) receiving a customer order over a data network at a controller that includes a data processor, the customer order identifying a Prescription ID associated with a compounded composition,
(b) receiving instructions from the controller at a processing station of a plurality of discrete stations, the processing station comprising a processing apparatus configured, in response to instructions received from the controller, to perform one or more process steps required for manufacturing the compounded composition, and the plurality of discrete stations further comprising a plurality of containers, and
(c) receiving instructions from the controller at an industrial robot configured, in response to instructions received from the controller, to displace one or more containers of the plurality of containers from one discrete station to another discrete station.

19. A non-transitory computer-readable medium having instructions in code which when executed by a processor of a controller causes the controller:
(a) receive a customer order over a data network, the customer order identifying a Prescription ID associated with a compounded composition,
(b) communicate instructions to a processing station of a plurality of discrete stations, the processing station comprising a processing apparatus configured, in response to instructions received from the controller, to perform one or more process steps required for manufacturing the compounded composition, and the plurality of discrete stations further comprising a plurality of containers,
(c) communicate instructions to an industrial robot configured in response thereto to displace one or more containers of the plurality of containers from one discrete station to another discrete station.
